(19) United States
(12) Patent Application Publication
(10) Pub. No.: US 2018/0080899 A1
(43) Pub. Date: Mar. 22, 2018

(54) FIXED AREA ELECTRODE FOR ELECTROCHEMICAL ANALYSIS OF HIGH TEMPERATURE FLUIDS

(71) Applicant: UNIVERSITY OF UTAH RESEARCH FOUNDATION, Salt Lake City, UT (US)

(72) Inventors: Michael Simpson, Salt Lake City, UT (US); Devin Rappleye, Millcreek, UT (US); Kevin Teaford, W. Jordan, UT (US)

(21) Appl. No.: 15/711,894

(22) Filed: Sep. 21, 2017

Related U.S. Application Data
(60) Provisional application No. 62/398,421, filed on Sep. 22, 2016.

(51) Int. Cl. G01N 27/48; G01N 27/36; G01N 33/20

(52) U.S. Cl. CPC G01N 27/48; G01N 33/206; G01N 27/36

(57) ABSTRACT

A method of making an electroanalytical measurement including providing a glass-fused working electrode that includes a tungsten rod and a glass coating fused to the rod to form a leak-proof seal, which defines a fixed working electrode area; inserting the electrode into a molten salt such that the fixed working electrode area is completely submerged and a portion of the glass coating is in contact with the molten salt; applying a potential to the molten salt; measuring current as a function of potential; and quantifying a concentration of an analyte.

### CROSS-REFERENCE TO RELATED APPLICATIONS

This application claims the benefit of U.S. Provisional Application No. 62/398,421 filed on Sep. 22, 2016, herein incorporated by reference in its entirety.

### STATEMENT REGARDING FEDERALLY SPONSORED RESEARCH

This invention was made with government support under grant number DE-NE0008310 awarded by the U.S. Department of Energy. The government has certain rights in the invention.

### TECHNICAL FIELD

The present invention pertains to methods of making electrochemical measurements. More particularly, the invention pertains to making electrochemical measurements in high-temperature molten salts using a fixed area electrode.

### BACKGROUND

Voltammetry can be an accurate, in situ technique to measure the concentration of ions in high-temperature molten salts, such as eutectic LiCl—KCl, which is used for the electrorefining of used nuclear fuel; the electrolysis of lithium, magnesium, or titanium; and the recycling of rare-earths. However, one of the greatest challenges in making accurate concentration measurement with voltammetry is determining the surface area of the working electrode (WE). In many earlier disclosures, the method of measuring the surface area of the WE is not discussed in detail or explicitly stated, which results in an unknown uncertainty of the measured WE surface area.

The most common and simple method is to observe a frozen salt layer or residue on a WE after exposure to the molten salt. The length of the salt layer on the WE is used with the diameter of the WE to calculate the area of the electrode-salt interface. The simplicity of this method results in a less accurate WE surface area due to surface tension effects (i.e. wetting, etc.) and observational errors.

In most cases, electrochemical measurements in molten LiCl—KCl eutectic are performed in an opaque furnace preventing the visual confirmation of the WE surface area in the salt. While a transparent furnace can be built and the WE immersion depth in the salt can be visually measured in the salt using a cathetometer, doing so would be extremely difficult and impractical to do, especially on an industrial scale.

One possible way to avoid the visual confirmation of the WE area is to use a differential area rather than an absolute surface area. This method performs an electrochemical technique at different increments of immersion depth. These increments can be made or measured precisely using a vertical position translator. The differential current with respect to the differential area can be related to concentration without the need to determine the absolute WE area. However, the precision of this method comes at the cost of a greater design complexity and longer turnaround time on concentration measurements due to the requirement to measure signals at multiple WE immersion depths.

Some have tried to use insulating material, such as alumina, boron nitride, or borosilicate to fix the WE area at a known value. However, none indicate whether the WE was simply sheathed, cemented, fused, or affixed to the insulator by some other means, except one in which the word “sheathed” is used. In other disclosures, the word “encased” is used. If the WE was simply sheathed, this would allow molten salt to creep up the thin gap between insulator and the WE, possibly augmenting the fixed area of the WE. Similar creepage could occur due to imperfect seals created by binding the insulator to the WE with cement or some other paste.

In the present disclosure, fusing an insulator to the WE results in a leak-proof seal, creating a truly fixed WE area. A WE with a well-defined fixed surface area created by an inert insulating coating greatly simplifies the construction of an electroanalytical probe without sacrificing accuracy. This probe can be used to detect impurities, monitor ions of interest, and provide feedback for process control and optimization in high-temperature molten salts.

### SUMMARY

The present disclosure provides a method of making an electroanalytical measurement. The methods include providing a glass-fused working electrode comprising a tungsten rod having a first end and a second end and a glass coating beginning at a first distance from the first end and ending a second distance from the second end, where the glass coating is fused to the rod so as to form a leak-proof seal and where the distance from the first end to the beginning of the glass coating defines a fixed working electrode area. The methods further include inserting the electrode into a molten salt such that the fixed working electrode area is completely submerged in the molten salt and at least a portion of the glass coating is in contact with the molten salt, applying a potential to the molten salt, measuring current as a function of potential, and quantifying a first concentration of a first analyte.

Other aspects of the invention will become apparent by consideration of the detailed description and accompanying drawings.

### BRIEF DESCRIPTION OF THE DRAWINGS

FIG. 1 is a picture of GFWE A before (top) and after (bottom) polishing.

FIG. 2 is a picture (left) and an illustration (right) of an experimental setup.

FIG. 3 is a plot of cyclic voltammograms at 100 mV/s in LiCl—KCl (eutectic)-UCl₃ (0.5 wt %) at 773 K with an alumina sheath in contact with the molten salt (solid curve) and with the alumina sheath removed from the molten salt (dashed curve). WE=1.5 mm W rod, RE=1 mm W wire, CE=U metal in SS basket, and WE area=0.46 cm².

FIG. 4 is a plot of a cyclic voltammogram at 100 mV/s in LiCl—KCl (eutectic)-UCl₃ (0.5 wt %) at 773 K with glass coating on GFWE B (area=0.593 cm²) in contact with salt for 13.7 hr. RE=1 mm W wire and CE=U metal in SS basket.

FIG. 5 is a graph showing peak height at various immersion levels of GFWE A in molten salts having varying concentrations of UCl₃. Solid lines indicate linear fits before glass-W interface; the vertical dot-dashed line indicates the glass-tungsten interface; the horizontal dashed lines indicate average values after glass-W interface.

FIG. 6 is a graph showing peak current for U³⁺ reduction on GFWE B over a long-term test with key events marked by boxed letters: (A) first CV after initial anodic cleaning, (B) changes in immersion depth, (C) first CV after second anodic cleaning, (D) period of high-stability, (E) constant immersion depth, (F) computer goes to sleep, and (G) removal and reinsertion of GFWE B.

FIG. 7 is a graph showing the results of stability tests with GFWE A after UCl₃ additions at 100 mV/s. Dashed lines represent the average of the stable peak values, and the numbers in parenthesis in the legend represent the mix number.

FIG. 8 is a graph showing peak current vs. square root of scan rate (v) at various concentrations of UCl₃ with linear regressions. WE=GFWE A, T=773 K, and area=0.621 cm².

FIG. 9 is a graph showing peak current normalized by the square root of scan rate at each concentration (slopes from FIG. 11).

FIG. 10 is a pair of graphs showing the ψ-function calculated from measured U³⁺ reduction peaks (ψ=I/(AC√(Dn³F³v/RT))) at multiple scan rates for mix #10 (0.0236 mol/dm³) and #1 (0.362 mol/dm³) on the left and right, respectively. Potentials are adjusted for IR drop. Rᵤ=0.15Ω, WE=GFWE A, T=773 K, and area=0.621 cm².

FIG. 11 is a graph showing recorded peak heights (data points) with a slope from 0 to 50 mVs (solid lines) extrapolated to 250 mV/s for each concentration tested. WE=GFWE A, T=773 K, and area=0.621 cm².

FIG. 12 is a graph showing normal pulse voltammograms at various concentration of UCl₃. tₚ=0.3 s, tᵢ=15 s, and step=10 mV.

FIG. 13 is a graph showing diffusional current from NPV as a function of UCl₃ concentration, and fits of data with and without the migration term.

FIG. 14 is a plot of cyclic voltammograms at 100 mV/s. The inset shows U⁴⁺/U³⁺ redox peaks. T=773 K and area=0.621 cm².

FIG. 15 is a plot of a cyclic voltammogram at 100 mV/s in mixture #9 at potentials much more negative than the U³⁺ reduction peak. T=773 K and area=0.621 cm².

FIG. 16 is a series of photographs showing images of QFWE A before electrochemical testing (far left), after electrochemical testing (left center), after rinsing with ultrapure water (right center), and after polishing (far right).

FIG. 17 is a graph showing U³⁺ reduction peak height normalized by area and square root of scan rate. Data comes from this disclosure and two other sources.

FIG. 18 is a plot of uranium peak current after 2 hr at a temperature ranging from 500 to 600° C. The molten salt was eutectic LiCl—KCl containing 1 wt % UCl₃.

FIG. 19 is a normal pulse voltammetry plot that shows the current measured with a glass fused working electrode when potentials from −3.3V to −2.0V versus a Cl⁻/Cl₂ reference electrode were applied to a mixture of LiCl—KCl eutectic with 11 wt % UCl₃ and 1 wt % GdCl₃ at 500° C.

FIG. 20 is a chronoamperometry plot showing current measured with a glass fused working electrode as a function of time when a potential of −2.5 V versus a Cl⁻/Cl₂ reference electrode was applied to the mixture of LiCl—KCl eutectic with 11 wt % UCl₃ and 1 wt % GdCl₃ at 500° C.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

One aspect of the invention is a glass fused working electrode (GFWE). The GFWE is demonstrated herein for a eutectic LiCl—KCl molten salt containing UCl₃, although the invention is not limited to these specific molten salts, solutes, or analytes. The GFWE was validated with 3 sets of tests: (1) immersion, (2) stability, and (3) concentration correlation. The first test confirms the insulating properties of the glass in contact with molten salt while fused to an electrode by observing the behavior of cyclic voltammetry (CV) peaks at various immersion depths. The immersion depth was incremented by a vertical position translator. The second test identifies whether significant degradation of a quartz coating on the GFWE occurs over time by monitoring CV peak heights and shapes over a prolonged period of time. The purpose of the third set of tests is two-fold: (1) characterize the CV and NPV response over a range of U³⁺ concentrations and (2) test the stability of the glass coating at higher concentrations.

Another aspect of the invention is a method of making electrochemical measurements, particularly voltammetry measurements in molten salts. In certain embodiments, the method includes providing a glass-fused working electrode. Additionally, the glass-fused working electrode may be a tungsten rod having a first end and a second end. The tungsten rod may also have a glass coating beginning at a first distance from the first end and ending a second distance from the second end. The glass coating may be fused to the rod so as to form a leak-proof seal. In some embodiments, the glass coating may comprise, consists essentially of, or consist of a quartz-based #1 grading glass. The quartz-based #1 grading glass may comprise, for example, about 85 wt % SiO₂, about 10 wt % B₂O₃, and about 5 wt % Al₂O₃; any other component may be present in an amount of less than about 1 wt %. In other embodiments, the glass coating comprises, consists essentially of, or consists of a borosilicate glass. The distance from the first end to the beginning of the glass coating may define a fixed working electrode area. The fixed working electrode area may reduce the error associated with electrochemical measurements in molten salt.

The method further may include inserting the electrode into a molten salt. The molten salt may be anhydrous, and may comprise mixtures containing one or more of LiCl, KCl, NaCl, MgCl₂, CaCl₂, CsCl, SrCl₂, BaCl₂, RbCl, LaCl₃, PrCl₃, CeCl₃, GdCl₃, and/or YCl₃. In some embodiments, the molten salt may comprise eutectic mixture. For example, the molten salt may comprise an anhydrous LiCl—KCl eutectic mixture. In some embodiments, the molten salt may further comprise a metal chloride solute, or analyte salt. The metal chloride analyte may be selected from the group consisting of alkali, alkaline earth, rare-earth, actinide, lanthanide and transition metal chlorides. For example, the metal chloride analyte salts may include, but are not limited to, UCl₃, PuCl₃, LaCl₃, CeCl₃, NdCl₃, or GdCl₃. The molten salt may comprise up to about 20 wt % metal chloride analyte salts. In some embodiments, the molten salt may form greater than about 0 wt % and less than or equal to about 11 wt % anhydrous UCl₃. In some other embodiments, the molten salt may comprise up to about 12 wt % anhydrous UCl₃, PuCl₃, LaCl₃, CeCl₃, NdCl₃, or GdCl₃. The electrode may be inserted such that the fixed working electrode area is completely submerged in the molten salt. The electrode may also be inserted such that at least a portion of the glass coating is in contact with the molten salt.

The method further may include applying a potential to the molten salt and measuring current as a function of potential. The method further may include quantifying a first concentration of a first analyte. In certain embodiments, the method includes quantifying a second concentration of a second analyte. Additionally, the method may include quantifying a third, fourth, or fifth concentration of different third, fourth, and fifth analytes, respectively.

The temperature of the molten salt at the time of measurement is not limited other than to the extent that the temperature should be higher than the melting point of the salt or salt mixture and lower than the melting temperature of the electrode material or the glass coating. Preferably, the temperature should be lower than the annealing temperature of the glass coating. For example, the temperature of the molten salt may be less than or equal to about 1000, about 950, about 900, about 850, about 800, about 750, about 700, about 650, about 600, about 550, about 500, about 450, about 400, about 350, about 300, about 250, about 200, about 150, or about 100° C.

The glass coating may be chemically inert to the components of the molten salt at the temperature and the potential of the measurement. For example, the glass coating may be selected so as to not chemically react with the molten salt or a solute within the molten salt at the temperature and the potential of the measurement. Likewise, the glass coating may be selected so as to not substantially degrade after prolonged exposure to the molten salt, such as greater than about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, or about 25 hours. Substantially degrade can mean, for example, that the measurable width or length of the electrode changes, that there is visible cracking, chipping, or delamination of the glass coating, or that there is a detectable amount of leaching of elements from the electrode or the glass coating into the molten salt.

Preferably, the potential applied to the molten salt should be kept in a certain range to avoid damage of the electrode or glass coating and to avoid unwanted redox reactions of the molten salt. In the presence of UCl₃, for example, it is preferable that the potential be maintained such that it is not more negative than −0.8 V and not more positive than 1.1 V vs. U³⁺/U at 1 atm. In the case of metal chlorides, it is generally desirable that the electrode is not chronically exposed to metal chlorides having an equilibrium potential more positive than 1.1 V vs. U³⁺/U at 1 atm. It is also generally desirable for a metal chloride salt to have an equilibrium potential more negative than about 1.1 V vs. U³⁺/U at 1 atm. Preferably, the molten salt is substantially free of UCl₄. Substantially free may mean less than about 0.1 mol %.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s)”, “include(s)”, “having”, “has”, “can”, “contain(s)”, and variants thereof, as used herein, are intended to be open-ended transitional phrases, terms, or words that do not preclude the possibility of additional acts or structures. The singular forms “a”, “and”, and “the” include plural references unless the context clearly dictates otherwise. The present disclosure also contemplates other embodiments “comprising”, “consisting of”, and “consisting essentially of”, the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrase “at least one of A, B, . . . and N” or “at least one of A, B, . . . N, or combinations thereof” are defined in the broadest sense to mean one or more elements selected from the group comprising A, B, . . . and N, that is to say, any combination of one or more elements A, B, . . . or N including any one element alone or in combination with one or more of the other elements, which may also include, in combination, additional elements not listed.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4”. The term “about” may refer to plus or minus 10% of the indicated number. For example, “about 10%” may indicate a range of 9% to 11%, and “about 1%” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

It should be kept in mind that the following described embodiments are only presented by way of example and should not be construed as limiting the inventive concept to any particular physical configuration.

### Examples

### Development of the Working Electrode (WE)

The WE was developed by using a quartz-based #1 grading glass (GS-10, Transition Glass Products Ltd., 84.8% SiO₂ 10.0% Bi₂O₃ 4.7% Al₂O₃ 0.45% BaO 0.01% Fe₂O₃) to coat a tungsten rod. The tungsten was high grade, clean, and polished without any pitting, corrosion, or foreign matter attached. The easiest seals were achieved with a tungsten rod having a diameter between 0.76 mm and 2.03 mm. In this case, tungsten rods (99.95%, Alfa-Aesar) 30 cm in length and 1.5 mm in diameter were used. A hydrogen and oxygen flame was set fairly sharp, and the tungsten was washed with the flame. When the tungsten reached the necessary temperature, the tungsten surface begins to pool and appear wet. At this point, the #1 cane (grading glass) was drawn across the rod with both the cane and tungsten in the flame to the desired length; the tungsten surface must be pooling to properly fuse the glass and tungsten. In all cases, the glass was fused to the tungsten rod starting approximately 1 cm from one end of the rod and ending 5-7.5 cm closer to the other end. A tungsten rod with a glass coating fused to it is referred to as a glass fused working electrode (GFWE).

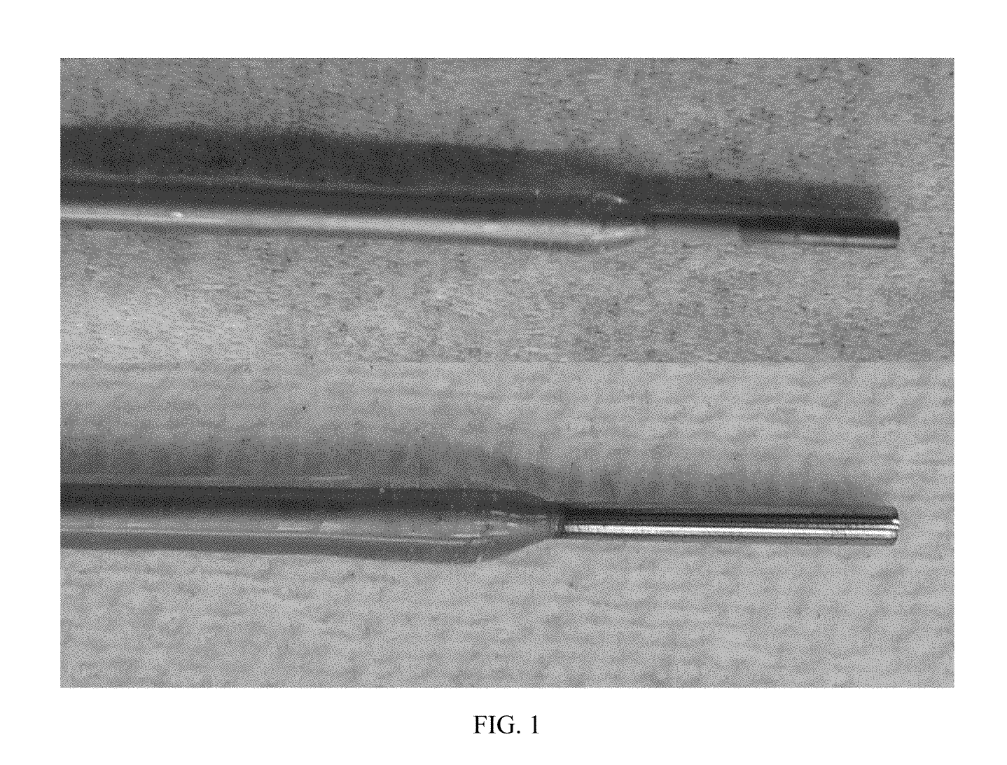

The glass blowing process exposed the tungsten rod to very high-temperatures that resulted in severe oxidation, as shown in FIG. 1. A yellowish green layer in the top image of FIG. 1 is indicative of tungsten (VI) oxide. The oxide layer was removed by polishing the tungsten portion of the GFWE with 2000-grit SiC paper until the surface was smooth and lustrous. After polishing, the diameter of the GFWE was verified to still be 1.5 mm using calipers. The length of the tungsten exposed beyond the glass coating was measured digitally and using calipers. The digital measurement used a photograph of the GFWE aligned with a square ruler and Plot Digitizer 2.6.6. The ruler was used as the axes to correlate pixels to distance. The distance was then calculated by marking the end of the tungsten rod and the tungsten-quartz interface. The exposed tungsten length was 12.8 mm for GFWE A and 12.2 mm for GFWE B. The respective estimated errors of the measured diameter and length measurements are 0.01 and 0.1 mm, resulting in less than 1.5% error in the WE area.

### Electrochemical Cell

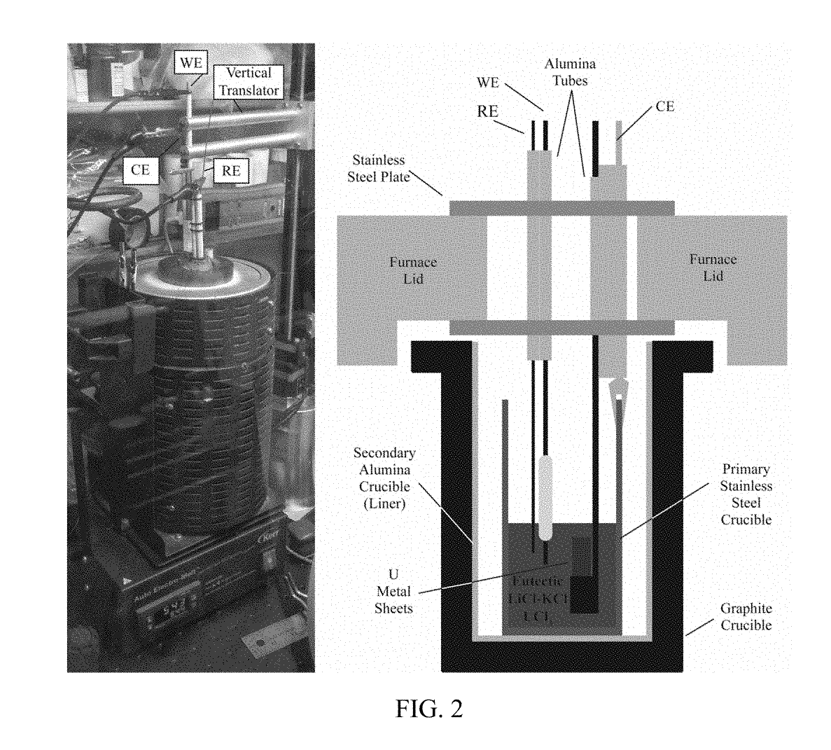

GFWE A and B were used in all experiments in the arrangement photographed and illustrated in FIG. 2. This setup was contained in an argon atmosphere glovebox (Inert Technology Inc., PL-HE-4 GB-2500) with H₂O and O₂ content below 0.5 ppm. The salt mixtures were heated in a Kerr Electro-melt furnace (Model No. 35224) to 500° C.±2° C. The interior of the furnace consisted of a graphite crucible in contact with a heating element. A 6 cm diameter alumina (99.6%) crucible (AdValue Technology, Part No. AL-2250) was inserted into the furnace to act as a liner in case of cracking in the primary crucible containing the molten salt. The primary crucible was a 316 stainless steel (SS) tube (1.5 in/3.8 cm OD, 3 in/7.6 cm long) and was disc welded together. A 5 cm diameter hole was drilled into the lid of the furnace. Two SS plates (Sharpe Products, Part No. 9445) were machined to have identical port holes that aligned precisely, preventing the tilting and swaying of electrodes. The two plates sandwiched the furnace lid and were attached using all-thread and nuts. Alumina tubes (McMaster-Carr) that fit snug in the holes were cemented in place on the top plate.

A pseudo-reference electrode (RE) was used and made of a 1 mm diameter W wire (99.95%, Alfa-Aesar) sheathed in an alumina tube (1.6 mm ID, 3.2 mm OD) until about 1-2 cm above the salt. The WE was rinsed in 3N HCl, polished by 2000-grit silicon carbide paper and anodically cleaned before conducting electrochemical tests. The WE was also sheathed in an alumina tube (3.2 mm ID, 4.7 mm OD) above the salt. The alumina sheaths of the WE and RE were bound together with two other alumina tubes (4.7 mm OD) and Viton O-rings (McMaster-Carr) in a circular arrangement. The alumina tubes electrically isolated the WE and RE and spaced the electrodes apart from each other.

Two types of counter electrode (CE) were used. One CE was a stainless steel rod and basket containing U metal sheets (International Bio-Analytical Industries Inc.). The U metal sheets came with a black layer that flaked off easily. The black layer was supposedly an oxide layer and was polished in the glovebox until the surface was silvery-gray. Another CE was the primary SS crucible with a SS rod attached as a lead. U metal from the SS basket was electrotransported to the SS crucible wall. This CE was used to verify that U metal in SS basket was a sufficiently large CE.

### Measurement Methods

Anhydrous UCl₃ was synthesized from depleted U metal by first converting it to a hydride with hydrogen gas, then exposing the hydride to dry HCl gas resulting in a dark red powder. UCl₃ was added to eutectic LiCl—KCl (99.99% anhydrous, SAFC Hitech) salt with U metal present to form mixtures of various compositions. Some additions of UCl₃ were made while the salt was still molten by using a custom made borosilicate funnel was fed through a port in the lid until it was 1-2 cm above the salt surface. The salt was then gently mixed by hand with a flat-end, SS stir rod for approximately 1 minute after the additions.

Cyclic voltammetry was then performed every 5 minutes until a stable, consistent response was observed. The actual concentration of UCl₃ in each eutectic LiCl—KCl mixture was determined by sampling of the salt followed by analysis via ICP-AES, listed in Table 1 for each mixture. Table 1 lists the concentration in two units for reference, but molar concentration is used throughout the specification.

TABLE 1
Composition of UCl₃ in LiCl—KCl eutectic

| | Mix No. | | | | | | | | | |
|---|---|---|---|---|---|---|---|---|---|---|
| | 1 | 2 | 3 | 4 | 5 | 6 | 7 | 8 | 9 | 10 |
| UCl₃ | 0.50%* | 0.99% | 1.51% | 2.21% | 2.57% | 3.12% | 3.53% | 4.45% | 5.83% | 7.31% |
| (wt %) | | | | | | | | | | |
| UCl₃ | 0.0236* | 0.0471 | 0.0719 | 0.1057 | 0.1231 | 0.1498 | 0.1701 | 0.2161 | 0.2857 | 0.3623 |
| (mmol/cc) | | | | | | | | | | |

*based on weighed salt amounts

Each mixture was analyzed electrochemically using cyclic voltammetry (CV) and normal pulse voltammetry (NPV) using a potentiostat (Autolab PGSTAT302N). CV was performed at scan rates ranging from 50-250 mV s⁻¹. At least 4 cycles were recorded at each scan rate to ensure repeatability. NPV was performed with a pulse time (tp) of 0.3 s, an interval time (ti) of 15 s and a step potential of 10 mV. The potential range for the pulse potential (Ep) in NPV was at least ±0.1 V from the equilibrium potential of U³⁺/U redox potential. The base potential (Eb) was 0.2-0.3 V more negative than the U⁴⁺/U³⁺ equilibrium potential. Only relative potential values are reported due to the use of a pseudo-RE.

### Selection of Insulator

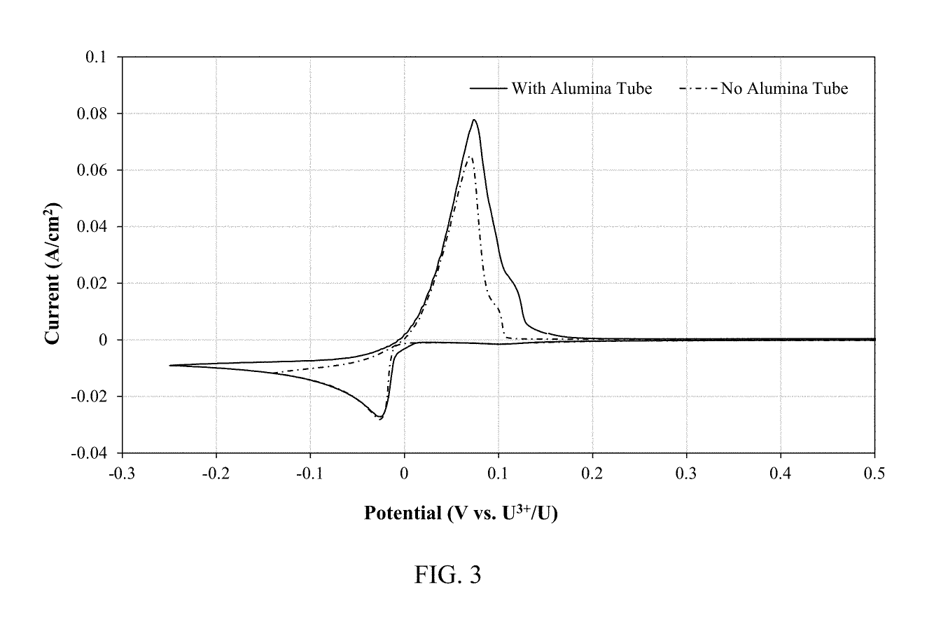

A ceramic, such as alumina or boron nitride, is difficult to seal to a metal substrate. A ceramic cement would not create a leak-tight seal. Even if a good seal was formed at room temperature, cracks could form in the cement or other adhesives when heated to temperature. Furthermore, cyclic voltammetry (CV) measurements were unintentionally made with the alumina sheath on the RE in contact with the molten salt in close proximity to the WE (3-5 mm). Upon recognition of this error the alumina was removed resulting in different peak heights and peak shapes, as shown in FIG. 3, indicating some possible interaction between alumina and the salt. No glass was in contact with the molten salt during these measurements. In FIG. 3, the U³⁺ reduction (negative current) peak has an additional wave before the peak when the alumina tube was in contact with the molten salt and the peak height is 3.2% lower.

Glass can be fused to tungsten to create a complete seal that can withstand the thermal cycling of the electrode from room temperature to the molten salt temperature. Borosilicate glass was considered because its coefficient of linear thermal expansion (3.3×10⁻⁶ K⁻¹) is similar to that of tungsten (4.5×10⁻⁶K⁻¹), but its low maximum service temperature (763-773 K) would limit its application. Quartz was targeted, but its thermal expansion coefficient (0.53×10⁻⁶K⁻¹) differed too greatly from tungsten's coefficient, which could possibly lead to cracking due to thermal stresses while heating. Therefore, the #1 grading glass, previously described, was used to ensure a good seal and compatible thermal expansion coefficients (1.05×10⁻⁶K⁻¹). Furthermore, its strain point (898 K) is 115 K higher than that of borosilicate glass (783 K), which represents the absolute maximum limit of serviceability for brief durations. This glass appeared to be durable with no visible signs of cracking or deformation after being heated gradually (25-35 K h⁻¹) from room temperature to 773 K four times.

### Electrochemical Behavior of UCl₃

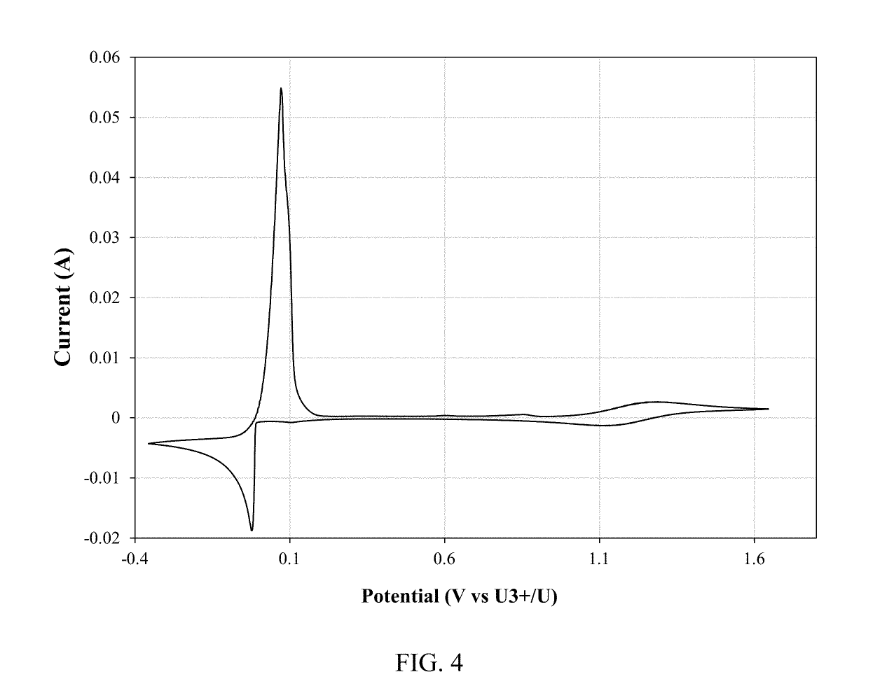

The cyclic voltammogram displayed in FIG. 4 was recorded after the glass coating had been in contact with the molten salt for more than 13 h and is characteristic of CV measurements made with the GFWE. Its features are consistent with CVs reported earlier, which were measured without a glass coating. These previous reports identified the prominent pair of peaks at approximately 0 V in FIG. 4 as the reduction of U³⁺ and oxidation of U metal. The sharp anodic peak and steep rise of the cathodic peak is characteristic of metal stripping and multiple electron transfer in a single step. The pair of peaks at 1.2 V has been previously identified as the oxidation of U³⁺ and the reduction of U⁴⁺. These broader peaks are characteristic of a single electron transfer reaction with a soluble reactant and product (soluble-soluble). A pair of peaks with a large spacing between them is also present in the cyclic voltammogram in FIG. 4, but due to their small size, the peaks are difficult to see. It is possible that these peaks are either the adsorption of uranium ions or the formation of a metal monolayer.

### Immersion Tests

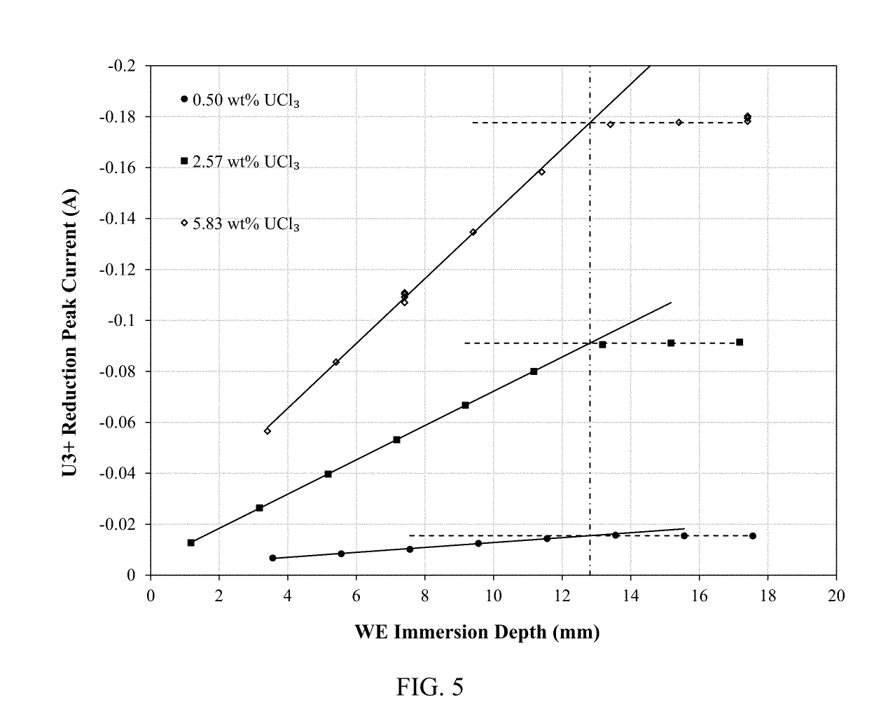

The initial test performed after heating the electrochemical cell to 773 K at 25-35 K h⁻¹ was the incremental immersion test. In this test, the position of the GFWE was adjusted using the vertical position translator. After adjustment, the surface tension on the WE was allowed to settle for two or more minutes, and then a CV measurement was made. The U³⁺ reduction peak heights at each position of GFWE A for mixture numbers 1, 5, and 9 are plotted in FIG. 5. There is a clear region where the peak height is linearly dependent on immersion depth, followed by a region where peak height is independent of immersion depth. The latter region indicates that the glass coating is indeed insulating the tungsten rod.

The position of the glass-tungsten interface was determined by fitting the linearly dependent peak heights (solid lines) in FIG. 5 and calculating the point at which the linear fit intercepted the average of the independent peak heights (horizontal dashed lines). This intersection was consistent at each concentration of UCl₃ and differed by a maximum of 2.1 mm from the expected depth; calculation of the expected depth has been previously observed to be ±2 mm due to surface tension effects.

### Stability Tests

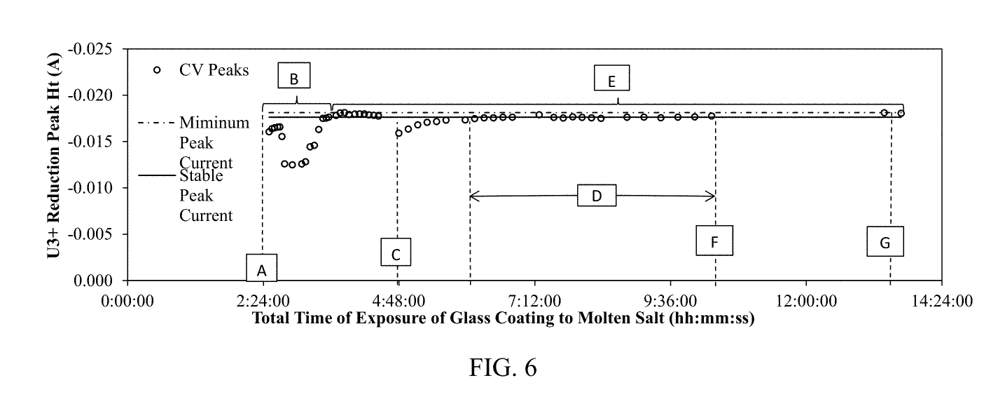

An initial stability test was performed on mixture #1 using GFWE B. FIG. 6 displays the temporal behavior of the U³⁺ reduction peak current for an 11-hour test with boxed letters indicating key events during the test. The time of the events are indicated by vertical, black dashed lines. The duration of some events are indicated by brackets at the top of the graph. Initially (event A), the GFWE was anodically cleaned by scanning the potential in the positive direction until tungsten dissolution. Immediately afterward (beginning of event B), the immersion depth of the GFWE was incrementally moved upward out of the salt. The current rises slightly, and then starts to drop significantly when the glass coating is removed from the salt. Then the GFWE was incrementally moved downward, resulting in the subsequent rise in the peak current. Once a clear plateau was established, the immersion depth of the GFWE was no longer adjusted (end of event B).

After fixing the vertical position of the GFWE (beginning of event E), a slight rise above the stable peak height value is observed in FIG. 6 followed by a slight decay. This decay was erroneously supposed to be due to the accumulation of a passivating layer on the GFWE surface, essentially blocking a small fraction of the GFWE. Thus the GFWE was anodically cleaned again by scanning up to the tungsten dissolution potential (event C). After cleaning, the peak current values in FIG. 6 returned to the values recorded after event A. Because the magnitude of current decreased after cleaning, it appears that a passivating layer was not formed. However, the monitoring of the peak heights at a fixed position revealed that some time is required for the conditions in the salt near the WE to equilibrate after scanning the potential to the dissolution of tungsten at the WE. This may be due to the oxidation of some U³⁺ to U⁴⁺ near the WE while the tungsten rod is being anodically cleaned. Applying a potential more negative than the U³⁺/U⁴⁺ equilibrium potential or stirring the salt may expedite the return to pre-cleaning conditions at the tungsten-salt interface.

After 1.5 h, the peak current in FIG. 6 settled to a stable (±1.9%) value indicated by the solid vertical line, which represents the peak current averaged over the period of high stability (event D). It also appears that the peak current was settling to the same stable value before the second anodic cleaning of the GFWE. This stable value was maintained for 4 h. At the 10 h and 20 min mark, however, the computer controlling the potentiostat went to sleep (event F) and cyclic voltammograms were no longer recorded. The experimental system was unmonitored at this point. After 3 h, the computer was turned back on and CV measurements were recorded again. The peak current was 2.5% more negative than the stable value, but was less negative than the most negative (minimum) peak value recorded at the 3 h and 50 min mark. This slight increase may have been caused by the absence of CV measurements conditioning the GFWE over the past 3 hours.

To test the repeatability of the GFWE-salt interface, GFWE B was removed from the salt then reinserted to the same position at the 13 h and 30 min mark (event G). After waiting 5 min, 4 cycles of CVs were run. The peak current recorded was in excellent agreement (0.3% error) with the previous peak height recorded. This indicates that the removal and replacement of the GFWE from the molten salt will not compromise the precision of the measurements. Thus the GFWE could be suspended above the salt in the hot zone when not recording measurements, potentially prolong its life by limiting its exposure in the molten salt bath.

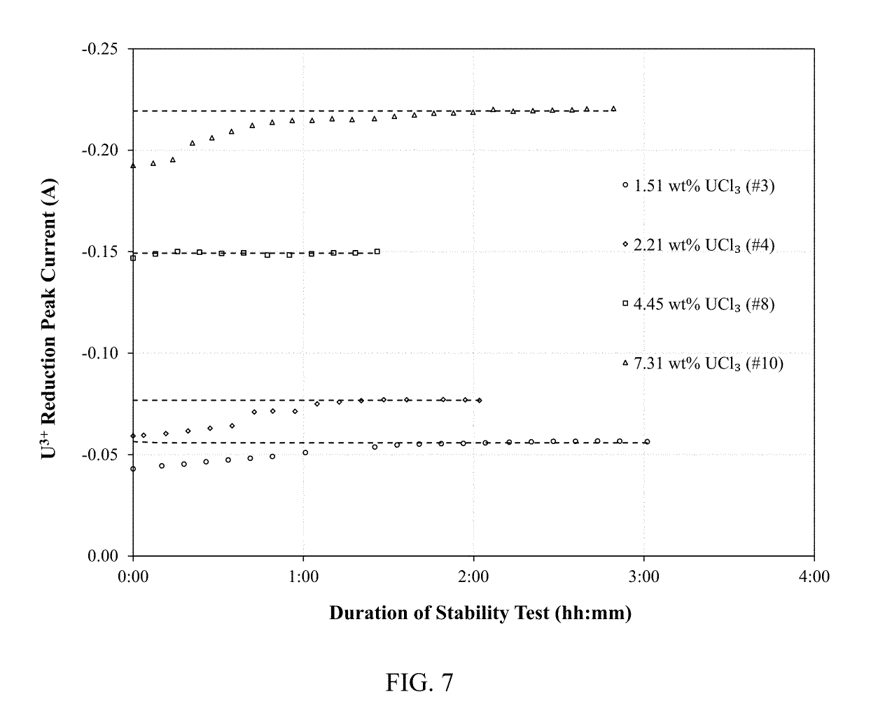

Additional stability tests were performed occasionally with GFWE A after additions of UCl₃ to ensure that the glass coating was not being degraded. The results of these stability tests are displayed in FIG. 7. The numbers in parenthesis correlate to the mixture number in Table 1. For comparative purposes, the x-axis in this plot displays the duration of the stability test, rather than the exposure time to the molten salt. Since UCl₃ was added while the salt was molten, there were two purposes to the stability tests: (1) ensure the integrity of the glass coating and (2) determine when the mixture has equilibrated after the addition of UCl₃ and stirring.

These tests were performed within 15-30 min of the addition of UCl₃. The gentle rise at the beginning of the stability tests in mixture numbers 3, 4, and 10 is likely to be due to the equilibration of thermal and concentration gradients in the salt mixture after adding cold UCl₃ powder. Mixture #8 may have reached equilibrium more quickly due to better initial mixing since stirring after each addition was done by hand and may have been inconsistent. The system was considered to be stabilized when the peak current stopped climbing. At this point, the peak current was averaged over at least an hour and compared to all U³⁺ reduction peaks recorded. After stabilizing, the peak current measured agreed within ±1.58%, 1.67%, 1.53%, and 1.03% of the average for mixture numbers 3, 4, 8, and 10, respectively.

### Cyclic Voltammetry Concentration Tests

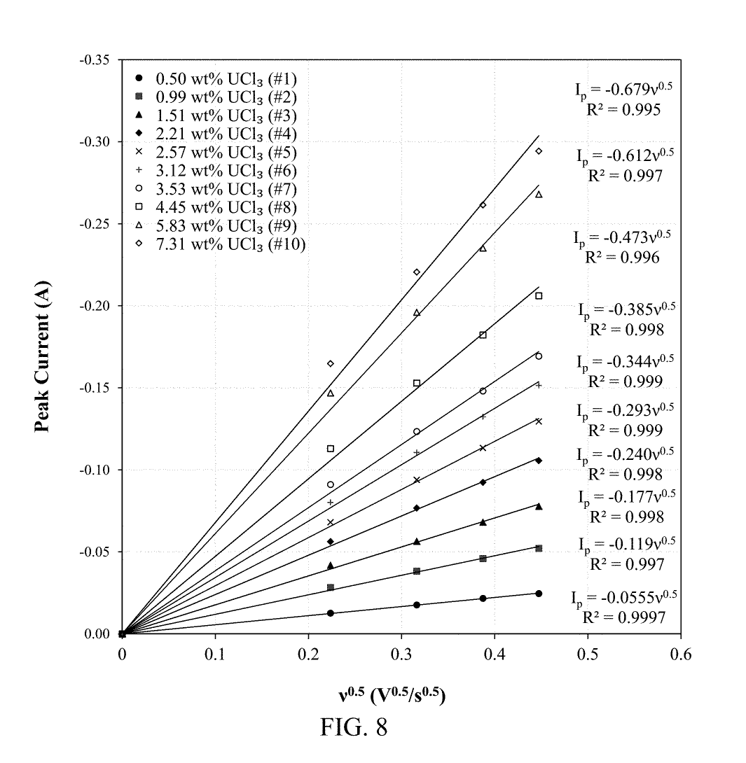

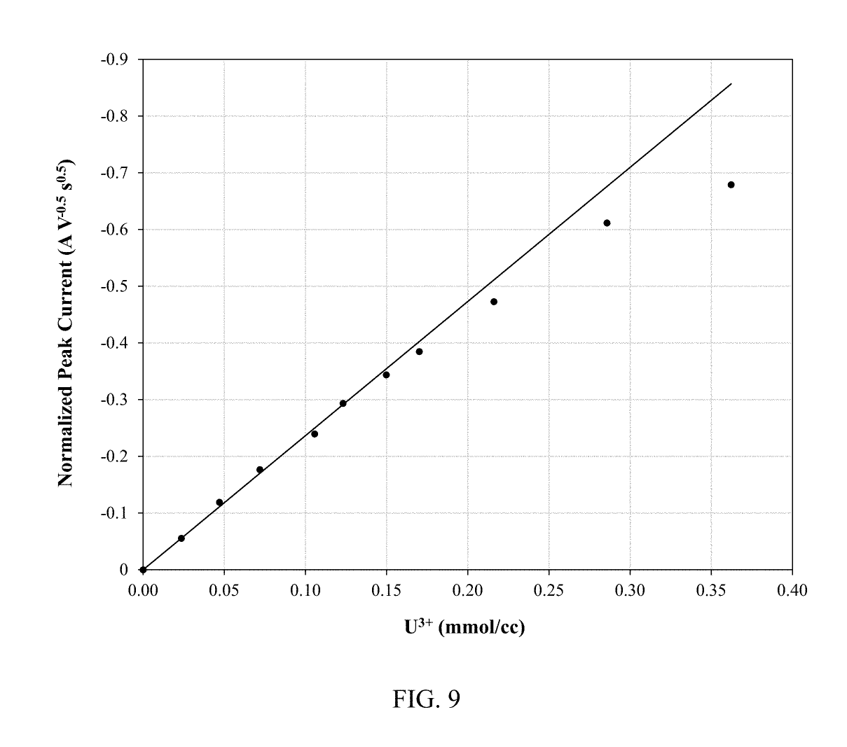

In FIG. 8, the U³⁺ reduction peaks recorded in CV at each concentration are plotted from 50 to 200 mV s⁻¹, which has been identified previously to be the limit of electrochemical reversibility for U³⁺ reduction. When an electrodeposition reaction is reversible, the Berzins-Delahay reaction is applicable to CV reduction peak:

$${I}_{p} = 0.6105 \sqrt{D \frac{{n}^{3} {F}^{3}}{RT} v} A C \qquad ( 1 )$$

where n is the number of electrons transferred, F is Faraday's constant, R is the universal gas constant, T is temperature, D is the diffusion coefficient, v is the scan rate, and C is concentration. Thus at each concentration, the peak current of U³⁺ reduction was regressed with respect to square root of scan rate as shown in FIG. 8. The slopes, called normalized peak currents, from FIG. 8 are plotted versus UCl₃ concentration in FIG. 9. The black line in the plot represents the linear trend regressed from 0 to 0.123 mol dm⁻³ UCl₃. The normalized peak current begins to depart significantly (>5%) from that trend after the 0.170 mol dm⁻³ UCl₃ data point. Possible explanations for this departure at higher concentrations could be diffusion layer growth, transition from planar to radial diffusion, counter electrode limitations, migration, transition from reversible to quasi-reversible behavior, or concentration dependence of the diffusion coefficient.

As UCl₃ concentration increased, the width of the U³⁺ reduction peak increased. This could be at least partially due to increasing ohmic (IR) drop (i.e. uncompensated resistance). Regardless of the cause of this broadening, more time was required to reach the peak. During this time, U³⁺ was actively reducing at the electrode. This increased duration of reduction may cause a gradual increase in the diffusion layer thickness due to depletion of ions. However, this phenomenon is accounted for in the CV theory.

Next, a possible transition from semi-infinite planar to radial diffusion was considered. Based upon the diameter (d) of the WE (0.15 cm) and the diffusion coefficient of U³⁺, the relative effect of radial diffusion is less than 5% when 4Dτ/d²≦3×10⁻³, where τ=RT/(nFv) for potential sweep methods. The diffusion coefficient of U³⁺ calculated from the linear regression in FIG. 9 is 1.09±0.0056×10⁻⁵ cm² s⁻¹. Thus, the scan rate (v) needed be greater than 14 mV s⁻¹ in order for peak current recorded with a cylindrical electrode to agree within 5% or less of a planar electrode. Furthermore, upon examination of the expressions for radial and planar diffusion, it was clear that radial diffusion would cause an increase in the absolute value of the current.

The CE limitation was also eliminated from consideration by using the SS crucible as the CE as described earlier. This effectively quadrupled the CE area, but the magnitude of the current only increased by 0.3%, which is within experimental error. Migration was also considered, but when a positively charged ion is being reduced, migration increases the magnitude of the current as well. However, there is evidence from previous spectroscopic studies that UCl₃ forms a [UClₓ]^{(x-3)-} complex in molten LiCl—KCl eutectic, where x has ranged from 5 to 7. However, the extent or strength of this complexation is questioned by some because electromotive force measurements of the U³⁺/U redox couple are in good agreement with thermodynamic calculations. If this complex is maintained in the presence of an applied electric field and migrates in this form, then the migration of the negatively charged complex could reduce the magnitude of the current as observed in FIG. 9. Further investigation of the migration behavior of U³⁺ in eutectic LiCl—KCl is planned and theoretical CV models accounting for migration may need to be developed.

Some have suggested that the bend in the uranium peak current at higher concentrations is due to a change in the diffusion coefficient. This is a plausible theory, but it should be validated via measurement of diffusion coefficients using a non-electrochemical approach. That is a non-trivial endeavor, however, and the resources for such a measurement were not available to us at the time.

The most likely explanation for the curvature in FIG. 9 is the onset and increase in charge transfer limitations. The rate of diffusion is dependent on the gradient between bulk and surface concentrations. As the bulk concentration increases, the limiting diffusional flux of U³⁺ is also increased. However, charge transfer is dependent on surface concentration. The reduction of ions at the WE surface decreases the surface concentration to zero, if diffusion limited, or some smaller value. Consequently, charge transfer rate may stay the same or increases to lesser degree because the surface concentration is increased at a slower rate than the concentration gradient near the WE. Eventually, the rate of charge transfer (i.e. reaction kinetics) could begin to have some control on the current density, rather than diffusion exclusively, as assumed in (1).

Others have formalized the theory and characterized the shape of CV peaks under the mixed control of diffusion and charge transfer, calling this condition quasi-reversibility. In their theory, the current is governed by a function, ψ, the shape of which is dependent on the transfer coefficient (α) and the parameter, Λ. The derived current is given by:

$$I = nFA \sqrt{D \frac{n F}{RT} v} C ψ ( E ) \qquad ( 2 )$$

where E is electrode potential and becomes the following equation at the peak:

$${I}_{p} = 0.447 A \sqrt{D \frac{{n}^{3} {F}^{3}}{RT} v} CK ( Λ , α ) \qquad ( 3 )$$

K is a tabulated function. The shape of the ψ-function is independent of scan rate at Λ-values greater than 15, below which the peak broadens and decreases in height until 10^{-2 (1+α)}. This peak broadening and shortening is indicative of quasi-reversible behavior (i.e. mixed diffusion and charge transfer control). The Λ-value is dependent on scan rate, as shown below and can be varied by adjusting the scan rate.

$$Λ = \frac{{k}^{o} {γ}_{ox}^{1 - α} {γ}_{red}^{α}}{\sqrt{{D}_{ox}^{1 - α} {D}_{red}^{α} ( \frac{n F}{RT} v )}} \approx \frac{{k}^{o}}{\sqrt{{D}_{ox}^{1 - α} {D}_{red}^{α} ( \frac{n F}{RT} v )}} \qquad ( 4 )$$

Thus as the scan rate increases, the Λ-value decreases.

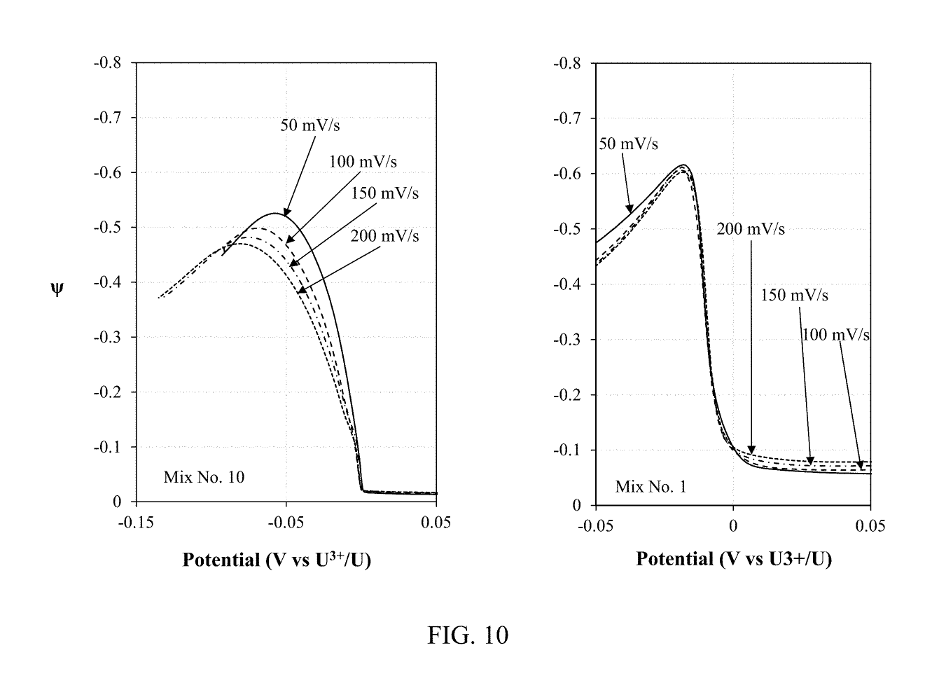

The ψ-function is calculated from the measured U³⁺ reduction current by dividing by the leading terms (n=3, D=1.09×10⁻⁵ cm² s⁻¹, T=773 K, A=0.621 cm²) in (2). This function is plotted in FIG. 10 for the lowest (mix #1) and the highest (mix #10) concentrations of UCl₃ at four different scan rates. Peak broadening may be caused by uncompensated resistance, Rᵤ. Therefore, the potentials reported in FIG. 10 have been adjusted for IR drop. As can be seen in FIG. 10, the U³⁺ reduction peak shape and height is independent of scan rate in mixture #1, but the peak becomes more broad and lower as the scan rate increases (Λ-value decreases) in mixture #10, indicative of quasi-reversible CV behavior. In fact, the height and the width of the ψ-function in mixture #10 at 50 mV s⁻¹ are lower and more broad than the ψ-functions in mix #1. The minima of the ψ-function also reaches −0.609 on average (std. dev. of 0.0054) in mixture #1, which corresponds closely with the leading constant of 0.6105 in (1), indicating that (3) simplifies to (1) at the minima when under diffusion control.

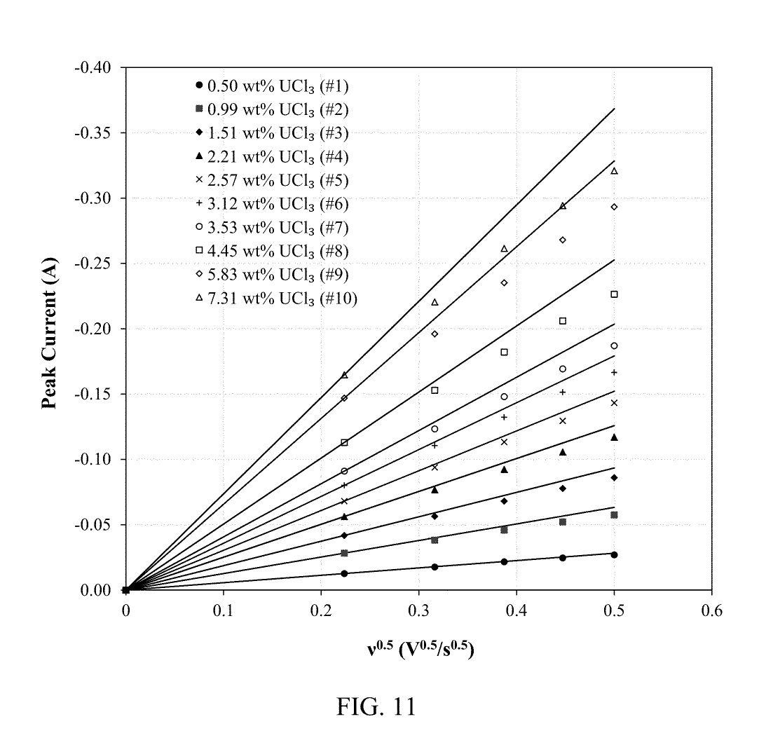

The decrease in magnitude of the minima of the ψ-function with increasing scan rate occurred in other high concentration mixtures, but is not directly evident in FIG. 8. However, a tilt in the peak height can be observed as the concentration increases. This tilt is observed by noting in FIG. 8 that as concentration increases, the peak heights drift further above and below the linear regression at lower and higher scan rates, respectively. A clearer demonstration of the shift in the transitional scan rate is displayed in FIG. 11, which includes the peak heights over the full range of scan rates tested (50-250 mV s⁻¹). The solid lines in FIG. 11 are the slopes calculated from 0 to 50 mV s⁻¹ for each concentration extrapolated to 250 mV s⁻¹. Theoretically, if U³⁺ reduction is reversible up to 200 mV s⁻¹, the slope from 0-50 mV s⁻¹ should be identical to slope from 50-200 mV s⁻¹, and the measured data points in FIG. 11 should lie close to the solid lines. However, as concentration increases, the peak heights begin to drift away from solid lines at lower scan rates. This increasing separation between the data points and the solid lines is additional evidence that charge transfer is beginning to influence the shape of the reduction peak because CV peak heights in the quasi-reversible region do not maintain linearity with the square root of scan rate.

From a theoretical standpoint, the limitation of the charge transfer step at higher concentration is unexpected. The parameter, Λ, in (3) was derived to determine if the electrochemical reaction is limited by diffusion (reversible), charge transfer (irreversible), or both (quasi-reversible). If Λ≧15, then the redox reaction is electrochemically reversible. However, Λ is commonly approximated with the expression on the right-hand side of (3), leading to the mistaken conclusion that if k^{o} and D are constant, then the scan rate at which a CV peak transitions from reversibility to quasi-reversibility is constant with concentration at the same temperature. However, the assumption that activity is equal to concentration is built into the right-hand side of (3). The complete expression in (3) with the γ terms relaxes that assumption for the charge transfer rate, but not for diffusion. Fick's second law is still simplified with the implicit assumption of the equality of activity and concentration. Hence, it becomes conceivable that k^{o} and D on the right-hand side of (3) could vary with concentration because activity coefficients are essentially embedded into the rate parameters.

Over a broad range of concentrations, the activity coefficient of U³⁺ could vary considerably, resulting in a change in k^{o} and/or D. A couple earlier studies have investigated the thermodynamic properties of UCl₃ in alkali chloride melts over a range of high (i.e. >0.095 mol dm⁻³ UCl₃) concentrations, and they indicate a decreasing activity coefficient with increasing UCl₃. Other earlier studies have evaluated the activity coefficient of UCl₃, but only report a single value, made measurements at a single concentration, and/or studied concentrations less than 0.095 mol dm⁻³ UCl₃. These findings indicate that the activity coefficients of U³⁺ do vary over the concentration range tested. This combined with FIGS. 10 and 11 support the theory that rate of charge transfer becomes more of a limiting factor at higher concentrations. This could be due to the concentration gradient near the WE increasing more rapidly than the WE surface concentration as more UCl₃ is added or due to changes in k^{o} and/or D with increasing UCl₃ content. Either or both of these explanations could be possible causes.

### Normal Pulse Voltammetry Concentration Tests

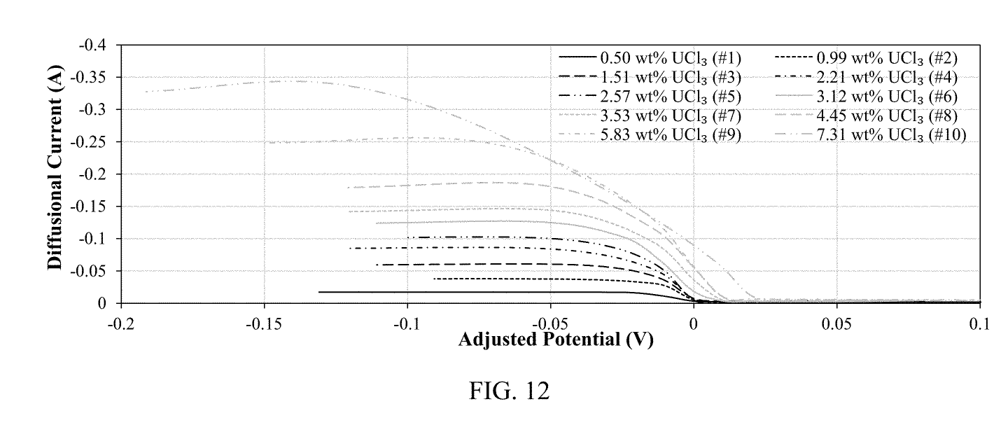

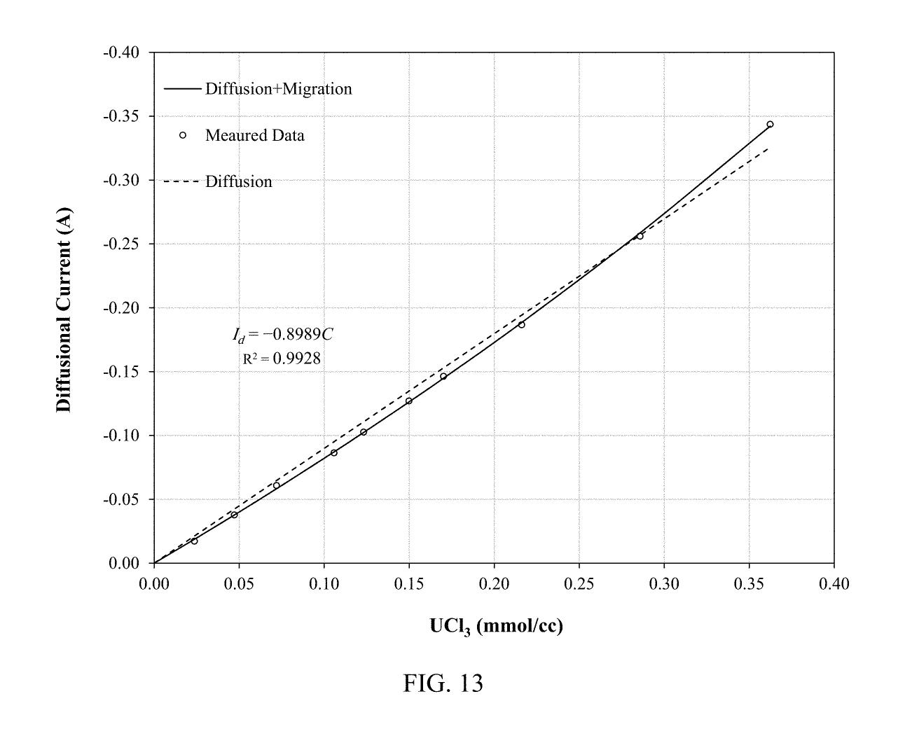

The behavior of the normalized peak current for CV in FIG. 9 is particularly perplexing in light of the normal pulse voltammetry (NPV) results. The NPV measurements made at each concentration are plotted in FIG. 12. The potential values in this plot are adjusted to align the NPV measurement for convenient comparison. At higher concentrations, the NPV plateau begins to decay at more negative potentials. It becomes visibly discernable above 0.216 mol dm⁻³ UCl₃. This decay is believed to be due to cumulative depletion of ions in the diffusion layer, which is caused by the use of a nonpolarographic (i.e. nonrenewing) electrode and the lack of stirring. Alternatively, the decay could be due to precipitation of U³⁺ ions near the WE if U³⁺ ions in excess of the solubility limit oxidize into the salt adjacent to the WE. In either case, ions are being depleted near the WE. Because ion depletion decreases the measured pulse current at more extreme potentials, the current value before the decay is used as the plateau or diffusional current. The diffusional current is plotted versus concentration in FIG. 13. The diffusional current was linearly regressed versus concentration. The resulting linear trend is plotted as the black, dashed line in FIG. 13. The diffusional current departs from linearity as well, but in the opposite direction (more negative) and less drastically.

At first glance, this deviation could be ascribed to WE area growth. However, the maximum charge passed during a pulse is 0.13 C at 0.362 mol dm⁻³ UCl₃, some of which is due to non-faradaic processes (i.e. double layer charging). At the same concentration, the charge passed before reaching the U³⁺ reduction peak in CV ranges from 0.12 C at 200 mV s⁻¹ and 0.19 C at 50 mV s⁻¹, and yet the peak heights in CV curve downward, rather than upward. Thus, if significant area growth occurs in NPV measurements, it would also occur in CV measurements. Another possibility is radial diffusion, but the criteria (Dt/r²≦3×10⁻³) for planar diffusion are met. Alternatively, the upward bend could be migration current that occurs due to potential gradients and contributes more significantly to the current adjacent to the WE at higher analyte concentration. Migration is neglected in most electroanalytical relations because they were derived for dilute analyte concentrations at which migration is negligible. Near the electrode and at non-dilute analyte concentration, the current recorded is given by the summation of the diffusional current (I_{d}) and migration current (Iₘ):

I=I_{d}+Iₘ (5)

The diffusional current in NPV is given by:

$${I}_{d} = \frac{nFA \sqrt{D} C}{\sqrt{π {t}_{p}}} = aC \qquad ( 6 )$$

If mass-transport and the electric field are linear then, the migration current is given by:

$${I}_{m} = \frac{{n}^{2} {F}^{2} ADC}{RT} \frac{Δ E}{l} = \frac{{n}^{2} {F}^{2} ADC}{RT} \frac{{IR}_{s}}{l} = bIC \qquad ( 7 )$$

where Rₛ is the solution resistance and l is the distance between the WE and CE. This results in the following expression for the total current:

$$I = \frac{nFA \sqrt{D} C}{\sqrt{π {t}_{p}}} / ( 1 - \frac{{n}^{2} {F}^{2} ADC}{RT} \frac{{R}_{s}}{l} ) = \frac{aC}{1 - bC} \qquad ( 8 )$$

where a and b are constants if the diffusion coefficient does not vary with concentration. Thus when reducing, migration current adds to the magnitude of diffusional current.

As shown in FIG. 13, the measured diffusional current fits (8) very well. Additionally, the diffusion coefficient calculated from a (D=1.79×10⁻⁵ cm² s⁻¹) is more consistent with the diffusion coefficient calculated from CV at concentrations≦0.123 mol dm⁻³ UCl₃ (D=1.09×10⁻⁵ cm² s⁻¹) than the diffusion coefficient calculated from a strictly linear fit (D=2.36×10⁻⁵ cm² s⁻¹) of the data in FIG. 13. Additionally, the resistance of the solution calculated from the b coefficient is 6.54Ω assuming a distance of 2 cm between the WE and CE. The estimate of 2 cm is based on the distance from the center of the WE port to the center of the CE port. The value of 6.54Ω is on the same magnitude as the resistance calculated (1.72Ω) based on the estimated distance from CE to WE, the WE area (0.621 cm²), and the specific conductance (1.872 Ω⁻¹ cm⁻¹) of molten LiCl—KCl eutectic. The difference may be due to a combination of factors, such as the complex geometry of the CE (i.e. uncertain cross-sectional area), the effect of U³⁺ ions on solution resistance, and uncertainty in the distance between electrodes. In any case, the parameters calculated from the a and b coefficient are promising evidence for migration.

Another way to evaluate the effect of migration is the transference number (t_{U3+}), which is the fraction of the total current carried by the migration of a given ion.

$${t}_{{U}^{3 +}} = \frac{{i}_{m , {U}^{3 +}}}{i} = \frac{\vert {z}_{{U}^{3 +}} \vert {u}_{{U}^{3 +}} {C}_{{U}^{3 +}}}{{Σ}_{k} \vert {z}_{k} \vert {u}_{k} {C}_{k}} \qquad ( 9 )$$

The mobility (u) of an ion is given by:

$$u = \frac{\vert z \vert FD}{RT} \qquad ( 10 )$$

where z is the charge of the ion. The diffusion coefficients of Li⁺, K⁺, Cl⁻ were determined in a previous molecular dynamics study at the eutectic composition from 638-1007 K and interpolated to be 1.9, 2.4, 2.5×10⁻⁵ cm² s⁻¹, respectively, at 773 K. The diffusion coefficient for U³⁺ calculated from NPV data is 1.79×10⁻⁵ cm² s⁻¹. Using these diffusion coefficients, the transference number for U³⁺ is calculated to be 4.2% at 0.362 mol dm⁻³, which suggests a non-negligible portion of the current is carried by U³⁺ ions migrating.

However, as mentioned, there is a possibility that U³⁺ could migrate as the [UCl₆]³⁻ complex, in which case migration would actually reduce the current at the WE during the reducing potential pulses. In this case, accumulation of U³⁺ ion near the WE due to repeated deposition and stripping of U metal on the WE could be the likely explanation. However, when optimizing the NPV waveform parameters at 0.198 mol dm⁻³ UCl₃, NPV measurements were recorded with a pulse time of 0.3 s and interval times of 10, 15, 20, and 30 s. The difference in the diffusional current at 10 s and 30 s was 0.9%. If significant amounts of ions were accumulating near the WE, it would be expected that the diffusional current would decrease significantly as the interval time is increased, because this would allow more time for ions to diffuse away from the WE. However, in order to more conclusively determine that a negligible amount of ions are accumulating next to the WE, NPV measurements are needed at higher concentrations, longer interval times (≧60 s), and/or with the rotation of the WE during the stripping of metal deposits.

The contrast in the NPV and CV data further supports the notion that the downward bend in the CV data (see FIG. 9) is due to the transition from diffusion to mixed (diffusion and kinetic) control. For example, if the diffusion coefficient decreased, a downward bend would also be reflected in the NPV measurements, because peak height in CV and diffusional current in NPV both depend on the square root of the diffusion coefficient. Indeed, it may be possible that the diffusion coefficient is actually increasing which, as shown in (4), would promote quasi-reversible behavior in CV and an upward bend in FIG. 13. Hence, variations in diffusion and activity coefficients cannot be eliminated as possible causes for the non-linear behavior of NPV and CV signals. Regardless, the transition to quasi-reversible behavior at higher concentration provides an explanation that is unique to the CV technique. This can reconcile the discrepancy in the CV and NPV results because the assumption of diffusion control was verified in NPV by the formation of the current plateau and the analysis of CV peaks at higher concentration (see FIGS. 10 and 11) revealed mixed diffusion and kinetic control. Therefore, the non-linearities in CV and NPV data in FIGS. 9 and 13 are likely caused by charge transfer limitations and migration, respectively. Migration probably affected CV measurements, but could not be observed due to the additional effect of charge transfer limitations.

Regardless of the explanation of the data, it is clear that non-ideal behavior is occurring at higher concentration and more work is needed to provide a definitive explanation of U³⁺ electrochemical behavior as concentration increases. This could provide substantial improvements in the accuracy of concentration measurements by electrochemical techniques. For example, if the upward curve in FIG. 13 is ignored and a simple linear regression is performed to fit the model in (6), then the 99.5% confidence interval (CI) on the predicted concentration is 0.036 mol dm⁻³. On the other hand, if migration is actually occurring and the model in (8) is valid, then the 99.5% CI is 0.0079 mol dm⁻³—a dramatic error reduction.

### Limitations

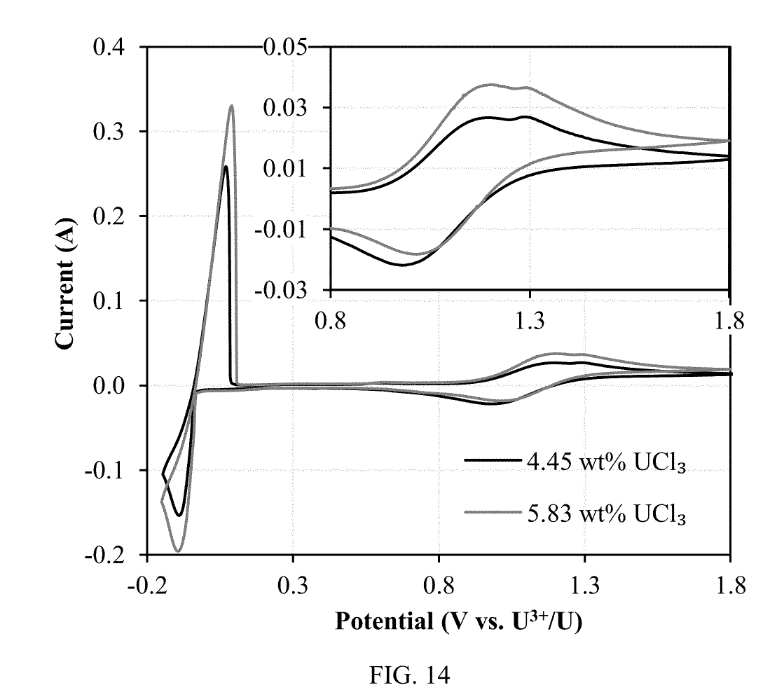

Two limitations to the application of the GFWE were elucidated in this study. First, at concentrations greater than 0.150 mol dm⁻³ UCl₃, a slight bump on the positive side of U³⁺ oxidation peak began to appear. This became more pronounced as the concentration of UCl₃ increased. It was quite evident at 0.216 mol dm⁻³ UCl₃ as shown in FIG. 14. The U³⁺ reduction peak appears unaffected. This shape of the CV in the inset of FIG. 14 is typical of an electrochemically reversible reaction followed by an irreversible chemical reaction. When an irreversible chemical reaction follows an electrochemical reaction, the peak in the CV after reversal decreases in magnitude more rapidly with scan rate than the peak preceding the chemical reaction. In this case, the U⁴⁺ reduction (negative) peak will reduce in magnitude more rapidly than the U³⁺ oxidation (positive) peak. Thus, the ratio of the cathodic (U⁴⁺ reduction) and anodic (U³⁺ oxidation) peak magnitudes would decrease with scan rate. However, no trend was observed. Additionally, the peak ratio (U⁴⁺ reduction:U³⁺ oxidation) from mixture #8 (1.14) is larger than the ratio from mixture #2 (1.04), in which no bump was observed, indicating that the reduction peak associated with the bump in FIG. 14 may be masked by the U⁴⁺ reduction peak. The oxidation and reduction of UO₂ occurs at a potential similar to the U⁴⁺/U³⁺ redox couple. Furthermore, it has been observed earlier that borosilicate glass reacts with UCl₄, which is less stable in LiCl—KCl eutectic than UCl₃. The following overall reaction is postulated as the possible explanation for the additional peak in FIG. 14.

UCl₄+SiO₂ [symbol] UO₂+SiCl₄

The standard Gibbs free energy of this reaction is 54.0 kJ mol⁻¹ resulting in an equilibrium constant of 2.24×10⁻⁴. Despite the small equilibrium constant, the reaction is still plausible, especially considering that high concentrations of UCl₄ would be present at the GFWE surface when UCl₃ is oxidized and the highly volatile nature of SiCl₄ (b.p.=58° C.). This volatility would further drive the reaction forward via La Chatlier's principle by its immediate vaporization and removal from the GFWE surface. Shortly after forming UO₂, the potential of the electrode is scanned to sufficiently positive potentials to oxidize UO₂, creating the bump in FIG. 14. Thus the formation of UCl₄ should be avoided, especially at higher concentrations of UCl₃.

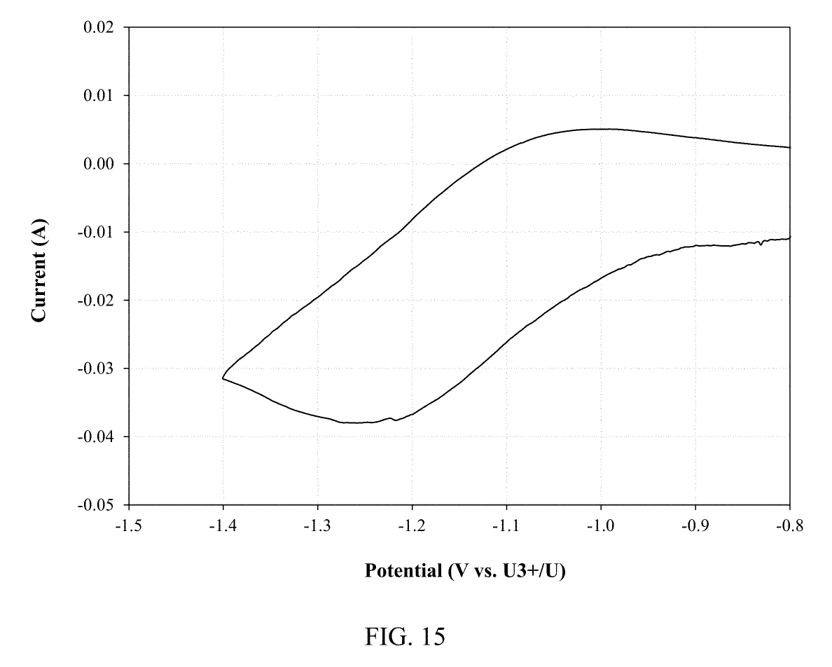

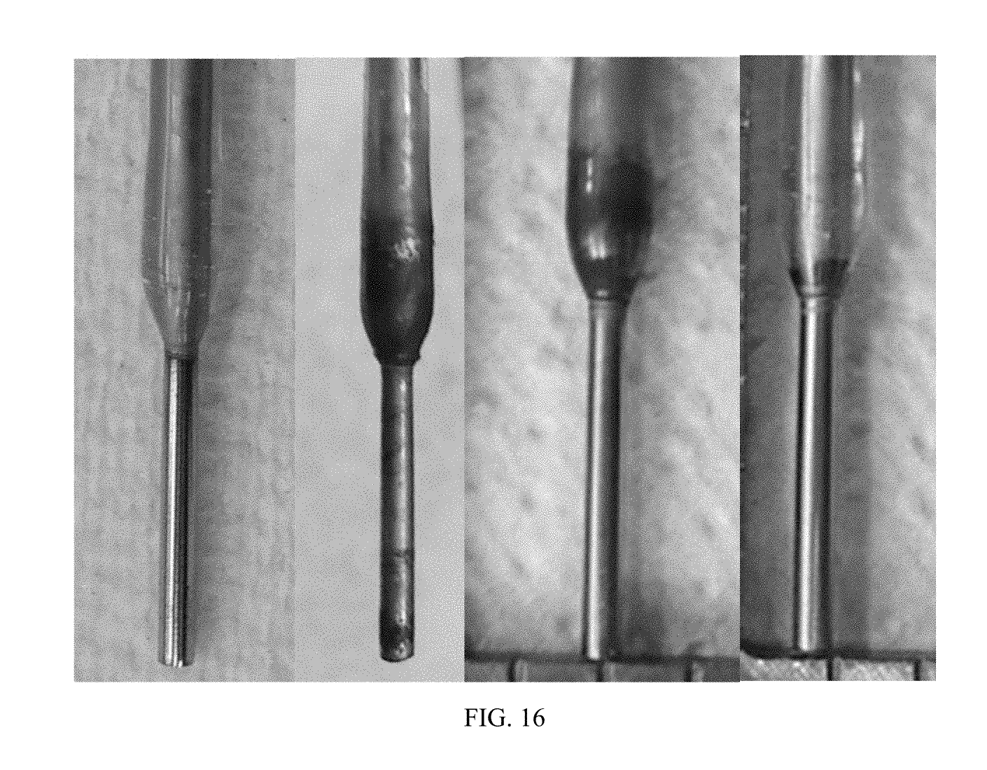

Next, the potential of the GFWE should not become more negative than −0.8 V vs. U³⁺(0.286 mol dm⁻³)/U. While performing CVs on mixture #9, the GFWE was briefly exposed to extremely negative potentials due to a momentary loss in stability of the pseudo-RE. A pair of peaks appeared at a potential similar to that reported (0.85 V vs. Ca²⁺/Ca) for the reduction of SiO₂ to Si in LiCl—KCl—CaCl₂ at 773 K, as shown in FIG. 15. After removing, cleaning, and polishing the GFWE A, a small patch of lustrous, metallic material was observed, as photographed in FIG. 16. The possibility of uranium was eliminated by scanning GFWE A with a Geiger-Mueller counter (Ludlum 26-1), which did not register any counts above background after rinsing GFWE A with water. Fortunately, pure silicon is also an insulator. It has a resistivity of 3.78×105 Ω cm, which is more comparable to silica (1.27×108 Ω cm) than tungsten (1.78×10-5 Ω cm) at 500° C. A multimeter (Fluke 115) measured the resistance of the glass coating, the lustrous grey layer, and tungsten. The glass and grey layer registered offline on the multimeter, indicating that the glass coating maintained insulating properties despite the possible formation of silicon. When applied to tungsten, the multimeter registered a resistance less than 1Ω. GFWE A was only exposed to these extreme conditions briefly, and the immersion tests, stability tests, and post-tests inspections (see FIGS. 5, 7, and 16) indicate that the integrity of the glass coating was not compromised.

### Data Comparison

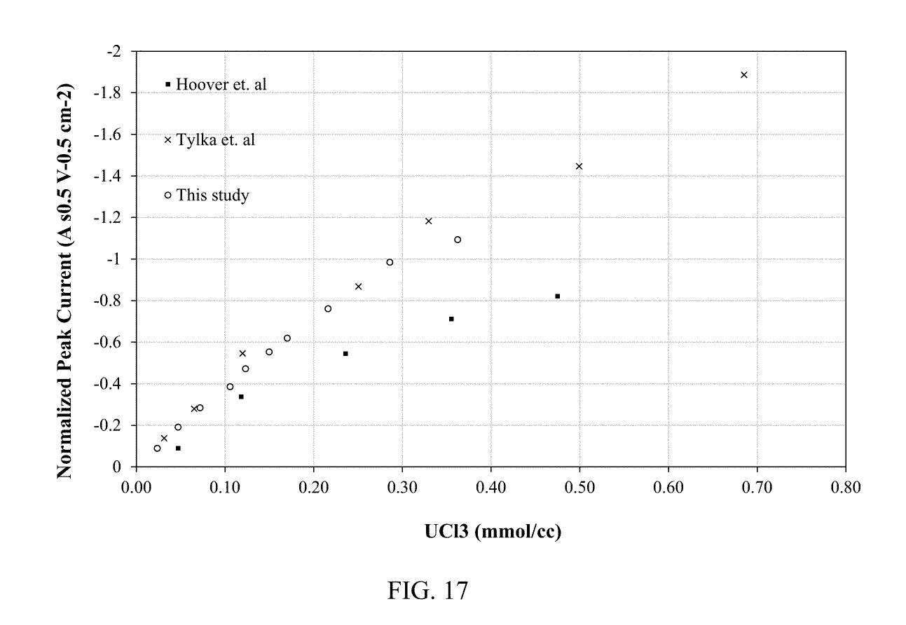

Further evidence for the integrity of the glass coating over the duration of these tests is gained by comparing the results of this disclosure to previous studies in which a bare tungsten rod was used as the WE. Two other studies have measured the U³⁺ reduction peak height in CV over a range of concentrations. The normalized peak height from each of the other two studies is overlaid with the values from this study in FIG. 17. At low concentrations, all three studies are in good agreement, but at higher concentrations the data reported by Hoover et. al departs significantly from Tylka et. al and this study.

### Temperature Stability

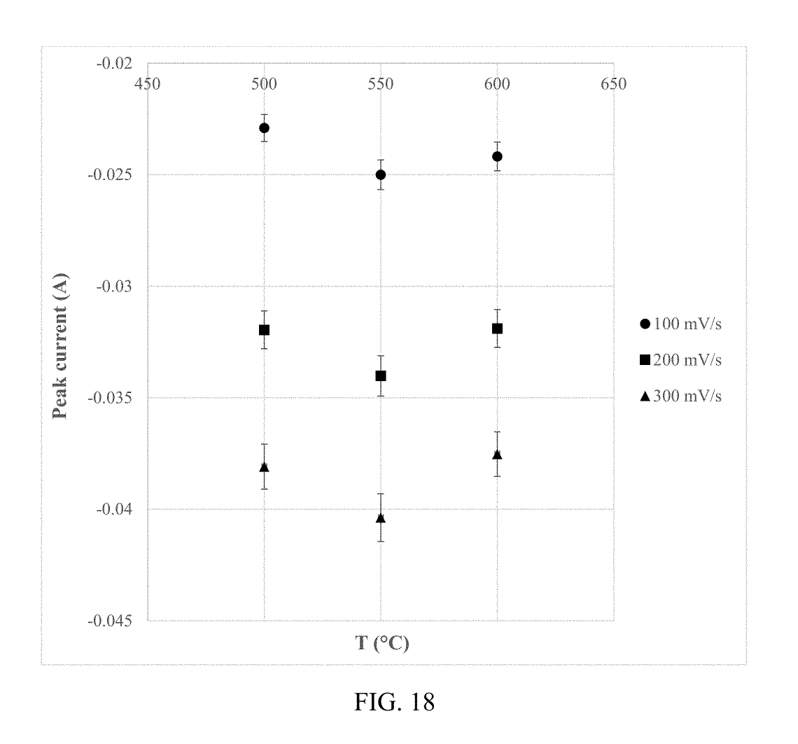

To test the stability of the glass coated electrode at varying temperatures, peak current was measured for a salt containing about 1 wt % UCl₃ in LiCl—KCl at 500, 550, and 600° C. at 0 and 2 hours after reaching each temperature. Average error was 2.65% between the initial and final peak measurement. The effect of temperature on the peak value for 3 different CV scan rates was plotted (FIG. 18). In each case, the peak values change within the estimated error bars. There appears to be no systematic change in peak current value as a function of temperature. Based on theory, the temperature should have caused the peak heights to decrease by 3% in going from 500 to 600° C. The average percent decrease in the peak current in going from 500 to 600° C. was 0.25%, supporting that the electrode works just as well at 600° C. as it does at 500° C. There is no measured loss in activity over 2 hour periods for any of the temperatures tested.

### Measurements of Multiple Analytes

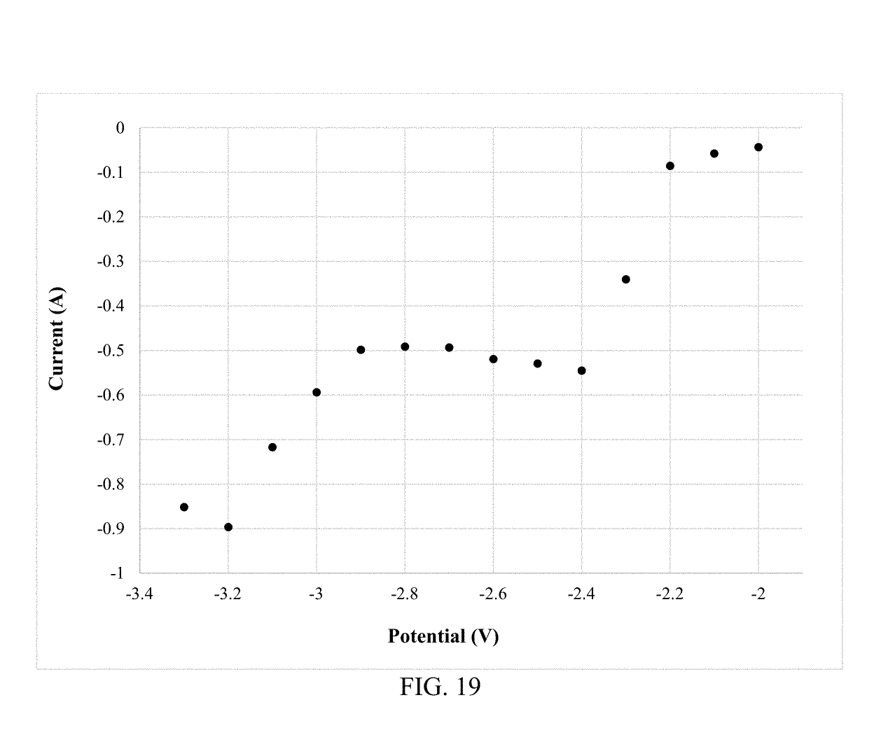

FIG. 19 is a normal pulse voltammetry plot that shows the current measured with a glass fused working electrode of the present disclosure when potentials from −3.3 V to −2.0 V versus a Cl⁻/Cl₂ reference electrode were applied to a mixture of LiCl—KCl eutectic with 11 wt % UCl₃ and 1 wt % GdCl₃ at 500° C. The two distinct current plateaus correspond to the UCl₃ and GdCl₃. FIG. 19 demonstrates the suitability of the glass fused working electrode to measure the current response of a high concentration of UCl₃ (i.e., 11 wt %) as well as to measure the current response of both UCl₃ and GdCl₃ in the same molten salt mixture. Additionally, the GdCl₃ can be measured at a relatively low concentration (i.e., 1 wt %) in a salt solution that includes a relatively high concentration of UCl₃. Because the GdCl₃ is a close electrochemical surrogate for PuCl₃, the electrode therefore may be used to measure the concentrations of both PuCl₃ and UCl₃ in molten salt solutions having small concentration of PuCl₃ relative to high concentrations of UCl₃. This functionality has specific application to measurement of molten salts used as nuclear fuels in which a small concentration of PuCl₃ exists compared to a high concentration of UCl₃. FIG. 19 also shows the electrode has an electrochemical range of at least 1.3 V.

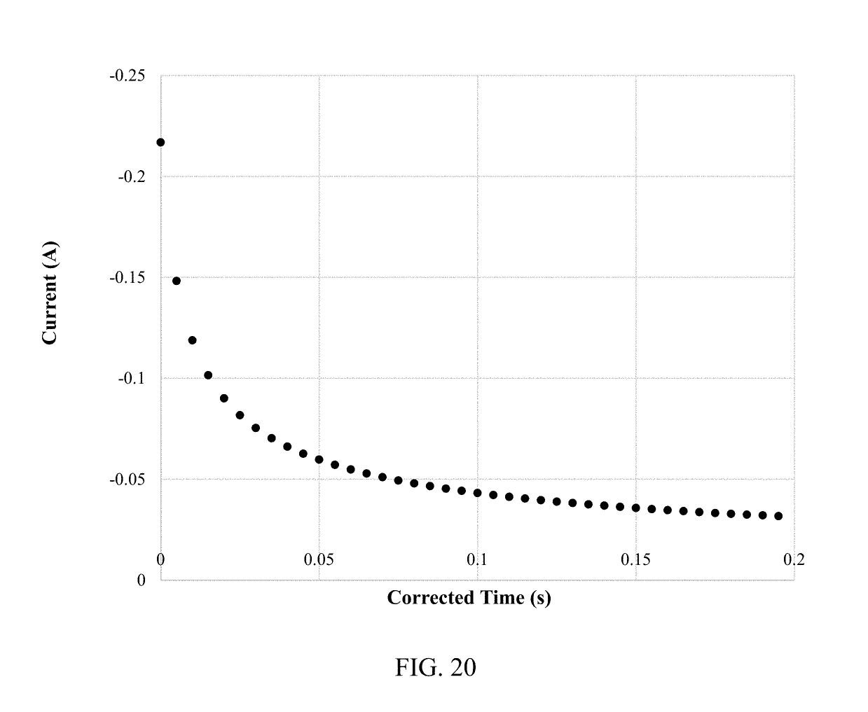

FIG. 20 is a plot showing the successful collection of chronoamperometry data using the glass fused working electrode of the present disclosure. Specifically, the glass fused working electrode was used to measure current as a function of time when a potential of −2.5 V versus Cl⁻/Cl₂ at 1 atm was applied to the mixture of LiCl—KCl eutectic with 11 wt % UCl₃ and 1 wt % GdCl₃ at 500° C. iR compensation was applied, which resulted in ideal Cottrellian behavior, even though the glass coating was inserted into the molten salt. Chronoamperometry is another method that can be used to determine the concentration of dissolved species in a molten salt.

Various features and advantages of the invention are set forth in the following claims.

What is claimed is:

1. A method of making an electroanalytical measurement comprising:
providing a glass-fused working electrode further comprising:
a tungsten rod having a first end and a second end; and
a glass coating beginning at a first distance from the first end and ending a second distance from the second end,
wherein the glass coating is fused to the rod so as to form a leak-proof seal,
further wherein the distance from the first end to the beginning of the glass coating defines a fixed working electrode area;
inserting the electrode into a molten salt such that the fixed working electrode area is completely submerged in the molten salt and at least a portion of the glass coating is in contact with the molten salt;
applying a potential to the molten salt;
measuring current as a function of potential; and
quantifying a first concentration of a first analyte.

2. The method of claim 1, wherein the glass coating comprises a quartz-based #1 grading glass.

3. The method of claim 2, wherein the temperature of the molten salt is less than or equal to about 600° C.

4. The method of claim 2, wherein the quartz-based #1 grading glass comprises about 85 wt % SiO₂, about 10 wt % B₂O₃, and about 5 wt % Al₂O₃, further wherein any other components are individually present in an amount of less than about 1 wt %.

5. The method of claim 1, wherein the glass coating comprises a borosilicate glass.

6. The method of claim 5, wherein the temperature of the molten salt is less than or equal to about 500° C.

7. The method of claim 1, wherein the glass coating is chemically inert to all components of the molten salt at the first potential.

8. The method of claim 1, wherein the electrode is not damaged by contact with the molten salt.

9. The method of claim 1, wherein the first potential is not more negative than −0.8 V vs. U³⁺/U at 1 atm.

10. The method of claim 1, wherein the first potential is not more positive than 1.1 V vs. U³⁺/U at 1 atm.

11. The method of claim 1, wherein the electrode is not chronically exposed to metal chlorides having an equilibrium potential more positive than 1.1 V vs. U³⁺/U at 1 atm.

12. The method of claim 1, wherein the glass coating does not substantially degrade when in contact with the molten salt for a period greater than about 25 hr.

13. The method of claim 1, wherein the molten salt comprises a metal chloride salt.

14. The method of claim 13, further wherein the metal chloride salt is selected from the group consisting of an alkali, an alkaline earth, a rare-earth, an actinide, a lanthanide and a transition metal chloride.

15. The method of claim 13, further wherein the metal chloride salt has an equilibrium potential more negative than about 1.1 V vs. U³⁺/U at 1 atm.

16. The method of claim 1, wherein the molten salt comprises an anhydrous LiCl—KCl eutectic.

17. The method of claim 1, wherein the molten salt comprises greater than 0 wt % and less than or equal to about 12 wt % anhydrous UCl₃.

18. The method of claim 1, wherein the molten salt comprises up to about 12 wt % anhydrous PuCl₃, LaCl₃, CeCl₃, NdCl₃, or GdCl₃.

19. The method of claim 1, wherein the molten salt is substantially free of UCl₄.

20. The method of claim 1, wherein the method further comprises quantifying a second concentration of a second analyte different from the first analyte.
